(19) United States
(12) Patent Application Publication
(10) Pub. No.: US 2020/0255801 A1
(43) Pub. Date: Aug. 13, 2020

(54) METHOD FOR THE IN VITRO PREPARATION OF DERMAL PAPILLA AND HAIR FOLLICLE EQUIVALENTS

(71) Applicant: L'OREAL, Paris (FR)

(72) Inventors: Khalid BAKKAR, Aulnay-Sous-Bois (FR); Theebah SELLATHURAI, Aulnay-Sous-Bois (FR)

(21) Appl. No.: 16/760,109

(22) PCT Filed: Oct. 30, 2018
(86) PCT No.: PCT/EP2018/079758

§ 371 Date: Apr. 29, 2020

(30) Foreign Application Priority Data
Oct. 30, 2017 (FR) 1760242

(51) Int. Cl. C12N 5/071; G01N 33/50; A61K 35/36

(52) U.S. Cl. CPC C12N 5/0628; C12N 2503/06; G01N 33/5044; A61K 35/36; C12N 5/0697; C12N 2506/1307; C12N 2513/00; C12N 2500/90; C12N 2500/32; C12N 2500/38; C12N 2500/34; C12N 2500/05; C12N 2501/39; C12N 2501/33; C12N 2501/999; C12N 2502/094; C12N 2502/09; C12N 5/0625

(57) ABSTRACT

The invention relates to a process for the in vitro preparation of a dermal papilla equivalent from fibroblasts derived from the dermal papilla and/or from the connective tissue sheath; to a process for the in vitro preparation of a hair follicle equivalent by culturing proliferative epithelial cells on said dermal papillae thus obtained; to the in vitro dermal papilla and hair follicle equivalents produced by means of the abovementioned processes, and to the uses thereof for treating alopecia and for evaluating the effect of cosmetic, pharmaceutical or dermatological products.

The present invention relates to a process for the in vitro the preparation of a dermal papilla equivalent from fibroblasts derived from the dermal papilla and/or from the connective tissue sheath.

The present invention also relates to a process for the in vitro preparation of a hair follicle equivalent by culturing proliferative epithelial cells on said dermal papillae thus obtained.

The invention similarly relates to the in vitro dermal papilla and hair follicle equivalents produced by means of the abovementioned processes, and to the uses thereof for treating alopecia and for evaluating the effect of cosmetic, pharmaceutical or dermatological products.

During its lifetime, the individual hair goes through three phases of development of very unequal duration:
The anagen phase is the active phase of the individual hair, during which it lives and grows regularly. It lasts from 4 to 7 years. The germ cells which surround the papilla of the root bulb of the individual hair continually produce the material which enables the individual hair to live and grow. Next, the multiplication of the germ cells stops at the bottom of the follicle. The active phase of the individual hair is then ended; another begins, which is much shorter.
The catagen phase: In barely 15 days, the bulb of the individual hair disappears (because it is no longer fed due to the interruption of the germ cells) and transforms at an accelerated rate. The papilla disappears, that is to say that the hollow bulb becomes solid; it keratinizes, hardens and becomes cornified. The individual hair is then dead; the follicle tightens so as to expel the dead individual hair.
The telogen phase is the phase which lasts approximately three months and during which the dead individual hair is waiting to fall out. In order to fall out, the individual hair must be pushed out by the new individual hair which in turn grows in the same follicle and which will expel the old hair.

The hair follicle then regenerates starting from the stem cells, known as germ cells, located in the “bulge”.

The “bulge” is formed by a cell subpopulation of the external epithelial sheath, known as germ cells, located in the middle portion of the hair follicle, and more exactly in the arrector pili muscle insertion zone. These cells represent the lowest part of the permanent portion of the follicle.

At the level of the “bulge”, the keratinocytes are biochemically and ultrastructurally relatively undifferentiated.

This “bulge” is in a strategic position for interacting, during the late telogen phase, with the ascending dermal papilla and initiating a new follicle in the anagen phase. The germ cells are therefore essential cells for renewal of the individual hair. The germ cells (germinative cells or pluripotent cells or stem cells) of the individual telogen hair are involved in the hair follicle leaving the dormant phase and therefore in the regrowth of the individual hair.

The hair bulb is pear-shaped and it is composed:
of the papilla which is a budding of dermal origin, located at the base of the follicle. It is a highly vascularized site which participates in the nutrition and regulation of the growth of the individual hair through its store of growth factors and extracellular matrix proteins. This information will be transmitted to the matrix cells, produced in the matrix, to allow differentiation thereof (Rees J L. Genetics of hair and skin color).
of the matrix which is a zone capping the dermal papilla, constituted of a clump of sparingly differentiated matrix cells. It is the site of intense mitotic activity. The matrix cells, which are located in the hair bulb and which form a small cell clump around the dermal papilla, are mainly constituted of precursors of keratinocytes which constitute a germinative stratum and which proliferate rapidly so as to differentiate to form the hair shaft, thus playing an essential role in the hair cycle. From the beginning of the anagen phase up to the end of said phase, these matrix cells will proliferate up to the catagen phase and then disappear in the telogen phase. (Ebling F J. The biology of hair. Dermatol. Clin. 1987 July; 5 (3): 467-81. Review; Saitoh M, Uzuka M, Sakamoto M. Human hair cycle. J. Invest. Dermatol. 1970 January; 54 (1): 65-81). Cell differentiation will allow the formation of the various cell types of the outer epithelial sheath (ORS), of the inner epithelial sheath (IRS) and then of the hair shaft. It is also this matrix which conditions the shape of the individual hair. The matrix is distributed uniformly about an axis of symmetry for straight hair, whereas it will be greater on one side for curly hair (Melissopoulos A. and Levacher C. Les annexes cutanées [The skin integuments]. In: La peau: structure et physiologic [The skin: structure and physiology], published by Lavoisier; 1998. pages 57-99). The matrix also comprises follicle melanocytes which are responsible for the pigmentation of the hair. The proliferation and differentiation of these matrix cells are controlled by the dermal papilla (Botchkarev V A, Kishimoto J. Molecular control of epithelial-mesenchymal interactions during hair follicle cycling. J. Invest. Dermatol. Symp. Proc. 2003 June; 8 (1): 46-55. Review);
of the outer and inner epithelial sheaths which are produced by the upper matrix of the hair bulb, also known as the keratinization zone. The outer epithelial sheath constitutes the outer envelope of the follicle: it is an invagination of the epidermis. It notably houses stem cells from which the hair follicle will be cyclically regenerated. The inner epithelial sheath separates the outer epithelial sheath from the hair shaft. This sheath is constituted of three cell types organized in keratinized concentric layers which accompany the growth of the individual hair. Henlé's layer, Huxley's layer and the cuticle which is formed from flattened cells, directed toward the hair matrix, are distinguished;
of the hair shaft which is partly visible, this is the hair. The structure of the hair shaft is made up of three distinct layers from the outside to the inside. There is the cuticle, the cortex and the medulla, all made up all keratinized cells.

Alopecia is conditioned by various factors: genetic, hormonal and environmental, by the diet and by physical activity. The hair has an essential esthetic and identity role.

Thus, healthy, strong hair and a dense head of hair throughout one's lifetime is a desire of most men and women.

Many techniques are known for treating alopecia, such as cell therapy, laser therapy or else implants without surgery. The latter gives an immediate result and is much less invasive than surgery.

In order to obtain implants, human hair follicles are obtained by culturing various cell types present in the hair bulb.

For several years, those skilled in the art have cultured, in vitro in 2D or 3D, hair follicle cells from the various compartments which make up the bulb. C. Higgins has shown that in in vitro 2D culture, the dermal papilla cells lose their induction capacity as soon as they are first cultured and amplified. The spheroids obtained in 2D and 3D culture described by Higgins do not have the functional features of in vivo dermal papilla: in particular, these spheroids have low activity toward alkaline phosphatase, Versican, and SFRP2 (secreted frizzled related protein 2) as shown in example 6 hereinbelow.

Other teams, such as Park in Korea, use a medium rich in amino acids and vitamins to form DPLTs (dermal papilla-like tissue). In particular, in vitro dermal papilla equivalents prepared from mesenchymal stem cells derived from umbilical cord and fibroblasts derived from dermal papillae are described, respectively, in the following documents WO 2009/014272 and US 2008/0145929. However, the use of mesenchymal stem cells originating from a biological sample of umbilical cord, described in WO 2009/014272, and the use of a 2D culture support treated for cell culture which does not prevent cell adhesion, described in US 2008/0145929, do not make it possible to obtain in vitro dermal papilla equivalents having the essential features of a dermal papilla in vivo, in particular from a morphological and/or functional viewpoint and notably positive alkaline phosphatase enzymatic activity.

Moreover, none of the abovementioned models contain proliferative epithelial cells such as the matrix cells or the germ cells, the presence and the amount of which are essential to the formation of a follicle structure.

WO 2017/055358 discloses the capacity of the matrix cells to regenerate a micro hair follicle in the presence of ROCK inhibitor. However, these matrix cells are seeded on fibroblasts which have lost their capacity to proliferate (3T3 fibroblasts, human fibroblasts which have been irradiated or treated with mitomycin), and not on dermal papillae. This thus entails functional and morphological differences as shown in FIG. 10 below.

There is thus a need for in vitro dermal papilla and hair follicle equivalents which have, respectively, most of the functional and morphological features of an in vivo dermal papilla or hair follicle, preferably a human hair follicle and notably one which is capable of regenerating.

Surprisingly, the Applicant has demonstrated, firstly, that the culturing of fibroblasts derived from the dermal papilla and/or from the connective tissue sheath in a serum-free medium on particular 2D or 3D culture supports of round-bottomed microplate type, these supports not permitting cell adhesion, makes it possible to obtain an in vitro dermal papilla equivalent which has most of the functional and morphological features of an in vivo dermal papilla.

Secondly, the seeding of proliferative epithelial cells such as matrix cells and/or germ cells on said in vitro dermal papilla equivalents obtained makes it possible to obtain an in vitro hair follicle equivalent which has most of the functional and morphological features of an in vivo human follicle, and notably one which is capable of regenerating.

Consequently, such dermal papilla and hair follicle equivalents prove to be particularly advantageous for studying the morphogenesis of a hair follicle, for predictive tests of the activity of cosmetic and/or pharmaceutical active agents and also for the prophylactic or therapeutic treatment of a state of reduced pilosity, and the treatment of alopecia.

Thus, according to a first of its subjects, the present invention relates to a process for the in vitro preparation of a dermal papilla equivalent, comprising at least one step of culturing fibroblasts derived from the dermal papilla and/or from the connective tissue sheath on a support comprising a serum-free nutritive culture medium B for a period of time that is sufficient to allow said fibroblasts to detach from said support and to group together to form at least one spheroid; the surface of said support used not permitting cell adhesion; said culture support is chosen from 2D or 3D round-bottomed microplate culture supports.

A second subject of the present invention relates to an in vitro dermal papilla equivalent which may be obtained according to the abovementioned process.

A third subject of the present invention relates to the use of a dermal papilla equivalent according to the invention and of proliferative epithelial cells such as the matrix cells and/or the germ cells, for preparing an in vitro hair follicle equivalent.

According to another of its subjects, invention relates to a process for the in vitro preparation of a hair follicle equivalent, comprising at least one step of culturing proliferative epithelial cells in the presence of at least one dermal papilla equivalent according to the invention for a period of time that is sufficient to allow differentiation of said proliferative epithelial cells into keratinocytes positive for the markers K85 and K35.

The present invention also relates to an in vitro hair follicle equivalent which may be obtained according to the abovementioned process.

The present invention also relates to the therapeutic uses of the hair follicle equivalent according to the invention, in the prophylactic or therapeutic treatment of a state of reduced pilosity, and the treatment of alopecia.

According to another of its subjects, the invention relates to the use of the hair follicle equivalent according to the invention for the in vitro testing of effects of active agents on the hair properties, and in particular for identifying a compound which modulates the growth of bodily hair and/or head hair.

The present invention also relates to a process for screening for compounds which modulate the growth of bodily hair and/or head hair, using the hair follicle equivalent according to the invention.

### DETAILED DESCRIPTION OF THE INVENTION

### Fibroblasts

For the purposes of the present invention, the term “fibroblasts derived from the dermal papilla” means fibroblasts taken from a microdissection of an anagen-phase hair follicle on the budding of dermal origin located at the base of the hair follicle.

Likewise, the term “fibroblasts derived from the connective tissue sheath” means fibroblasts taken from a microdissection of an anagen-phase hair follicle at the base of the hair follicle under the dermal papilla.

The fibroblasts derived from the dermal papilla or from the connective tissue sheath are preferably taken from surgical waste or biopsies, for instance facelift waste or a scalp sample, since simply pulling out the individual hair does not make it possible to recover these cells.

In particular, the fibroblasts used in the context of the present invention are not fibroblasts whose proliferation was previously stopped, preferentially by having previously irradiated them (for example with gamma rays) or having previously treated them with mitomycin. Preferably, the the fibroblasts according to the invention are not 3T3 fibroblasts.

### Proliferative Epithelial Cells

The term “proliferative epithelial cells” means epithelial cells which are present in the hair follicle and which are capable of giving rise to all the cell types present in an individual hair. The proliferative epithelial cells are preferably chosen from the matrix cells and/or the germ cells.

The term “matrix cells” means the cells located in the hair bulb and which form a small cell clump around the dermal papilla (Ebling F. J. The biology of hair. Dermatol. Clin. 1987 July; 5 (3): 467-81. Review; Saitoh M, Uzuka M, Sakamoto M. Human hair cycle. J. Invest. Dermatol. 1970 January; 54 (1): 65-81). Samples of these cells may be taken, and they may be amplified and stored in tissue libraries for the purpose of subsequent use.

In order to preserve the integrity of the matrix tissue, these cells may be sampled according to the process which follows: hair follicles are placed in a Petri dish containing a minimum culture medium supplemented with 2% of antibiotic and nonessential amino acids.

The bulb region is cut at the top of the dermal papilla and the epithelium of the bulb is thus separated from the dermal papilla and from the connective tissue sheath.

For the purposes of the invention, the term “germ cells” means the stem cells present in the “bulge”. Preferably, the germ cells are sampled in the telogen phase.

The purpose of the invention, the term “telogen” means the follicle which contained the individual hair in the telogen phase.

The germ cells may be sampled according to the process which follows: telogen-phase hair follicles are placed in a Petri dish containing a minimum culture medium supplemented with 2% of antibiotic and nonessential amino acids.

The region of the germ cells, known as the bulge, is extracted from the connective tissue sheath and is subsequently placed in a Petri dish containing a feeder layer of 3T3i fibroblasts.

This microdissection technique thus makes it possible to preserve the amount and the integrity of the cells, since they are not separated from one another.

### Process for Preparing a Dermal Papilla Equivalent

The present invention relates to a process for the in vitro preparation of a dermal papilla equivalent, comprising at least one step of culturing fibroblasts derived from the dermal papilla and/or from the connective tissue sheath on a support comprising a serum-free nutritive culture medium B for a period of time that is sufficient to allow said fibroblasts to detach from said support and to group together to form at least one spheroid; the surface of said support used not permitting cell adhesion; said culture support is chosen from 2D or 3D round-bottomed microplate culture supports.

The term “spheroid” means a 3D microtissue in the form of a sphere, in which the cells will become organized and will synthesize extracellular matrix. It is considered that said dermal papilla equivalent is obtained when at least one spheroid is seen to appear after performing the process according to the invention.

For the purposes of the present invention, the term “serum-free” refers to a culture medium comprising less than 5% by volume of serum, less than 4% by volume, less than 3% by volume, less than 2% by volume, less than 1% by volume, preferably 0% by volume of serum relative to the total volume of the culture medium.

The term “serum” means the serum proteins contained in serum, in particular at least one serum protein chosen from albumins, globulins such as alpha-1-globulins, alpha-2-globulins, beta-globulins, gamma-globulins, and mixtures thereof.

During said step of culturing the fibroblasts derived from the dermal papilla and/or from the connective tissue sheath, the formation of clusters is observed at at least 1 day, then said clusters form aggregates at at least 2 days. Finally, the appearance of spheroids is observed at at least 3 days.

For the purposes of the present invention, the term “clusters” means a 2D collection of cells. For the purposes of the present invention, the term “aggregates” or “early spheroid” means a 3D clump of cells of irregular shape, which is not organized and does not yet synthesize extracellular matrix.

In a preferred embodiment, said nutritive culture medium B comprises from 500 to 1500 mg/l of amino acids, from 2 to 18 mg/l of vitamins, from 1500 to 4500 mg/l of glucose, from 8750 to 10 000 mg/l of inorganic salts, from 2 to 20 μg/ml of insulin, from 2 to 60 ng/ml of hydrocortisone, and optionally from 50 to 200 μg/ml of antibiotics and/or of antimycotics.

Advantageously, said nutritive culture medium B comprises from 600 to 1250 mg/l of amino acids, from 5 to 15 mg/l of vitamins, from 1750 to 2250 mg/l of glucose, from 9000 to 9750 mg/l of inorganic salts, from 5 to 15 μg/ml of insulin, from 5 to 50 ng/ml of hydrocortisone, and optionally from 50 to 200 μg/ml of antibiotics and/or of antimycotics.

Better still, said nutritive culture medium B comprises from 700 to 1150 mg/l of amino acids, from 5 to 15 mg/l of vitamins, from 1750 to 2250 mg/l of glucose, from 9000 to 9750 mg/l of inorganic salts, from 5 to 15 μg/ml of insulin, from 5 to 50 ng/ml of hydrocortisone, and optionally from 100 to 180 μg/ml of antibiotics and/or of antimycotics.

The amino acids present in said medium B are preferably chosen from glycine, L-glutamine, L-alanine, L-arginine, L-asparagine hydrate, L-aspartic acid, L-cysteine, L-cystine dihydrochloride, L-glutamic acid, L-histidine, L-isoleucine, L-leucine, L-lysine hydrochloride, L-methionine, L-phenylalanine, L-proline, L-serine, L-threonine, L-tryptophan, L-tyrosine dihydrate sodium salt, L-valine, and mixtures thereof.

The vitamins present in said medium B are preferably chosen from ascorbic acid, biotin, choline chloride, calcium D-pantothenate, ergocalciferol, folic acid, menadione sodium bisulfate, niacinamide, pyridoxal hydrochloride, riboflavin, thiamine hydrochloride, retinyl acetate, vitamin B12, alpha-tocopherol phosphate disodium salt, i-inositol, and mixtures thereof.

The inorganic salts present in said medium B are preferably chosen from anhydrous calcium chloride (CaCl₂), copper sulfate pentahydrate (CuSO₄.5H₂O), ferrous sulfate heptahydrate (FeSO₄.7H₂O), anhydrous magnesium sulfate (MgSO₄), manganese sulfate monohydrate (MnSO₄.H₂O), potassium chloride (KCl), sodium bicarbonate (NaHCO₃), sodium chloride (NaCl), anhydrous sodium dihydrogen phosphate (NaH₂PO₄), zinc sulfate heptahydrate (ZnSO₄.7H₂O), and mixtures thereof.

Examples of antibiotics present in said medium B that may be mentioned include penicillin, streptomycin, and mixtures thereof.

An example of an antimycotic present in said medium B that may notably be mentioned is amphotericin B.

Said serum-free nutritive culture medium B used preferably comprises less than 15 μg/ml of growth factors, preferably less than 12 μg/ml of growth factors. In particular, the growth factors present in said nutritive medium B are chosen from insulin and/or hydrocortisone.

In a particularly preferred embodiment, said nutritive culture medium B comprises from 80% to 99% by volume of Williams medium E, from 250 to 350 mg/l of glutamine, from 2 to 20 μg/ml of insulin, from 2 to 60 ng/ml of hydrocortisone, and optionally from 50 to 200 μg/ml of antibiotics and/or of antimycotics. Better still, said nutritive culture medium B comprises from 95% to 98% by volume of Williams medium E, from 280 to 320 mg/l of glutamine, from 5 to 15 μg/ml of insulin, from 5 to 50 ng/ml of hydrocortisone, and optionally from 100 to 180 μg/ml of antibiotics and/or of antimycotics.

The Williams medium E is in particular free of glutamine

The fibroblasts are preferably cultured for at least three days, better still between 4 and 21 days.

The fibroblasts are preferably seeded at high density, in particular at a density of at least 3000 cells/cm², preferably at least 9000 cells/cm², better still at least 14 000 cells/cm², even more preferably at a density of between 14 000 cells/cm² and 47 600 cells/cm².

In a preferred embodiment, the fibroblasts are seeded at a density of between 23 500 cells/cm² and 24 500 cells/cm², better still at a density of 23 800 cells/cm².

The support not permitting the adhesion of cells, in particular of fibroblasts, is chosen from 2D and 3D round-bottomed microplate culture supports.

Advantageously, the 2D and 3D supports used in the context of the present invention are not collagen-coated.

Among the 2D culture supports, mention may notably be made of bacteriological Petri dishes not treated for cell culture, in particular made of plastic, not permitting cell adhesion.

The surface of said support is preferably neutral. For the purposes of the present invention, the term “neutral surface” means a non-charged surface.

The surface of said support may be hydrophobic or hydrophilic, preferably hydrophobic.

When said surface is hydrophilic, it is covered with a substance such that it prevents any cell adhesion; in particular, such a substance may be chosen from hydrogels covalently bonded to the surface of said support.

In a particularly preferred embodiment, the surface of said support is neutral and hydrophobic. A bacteriological Petri dish sold under the name Falcon® by the company Corning (reference: 351007) may be used as 2D culture support.

Among the round-bottomed 3D microplate culture supports, use may notably be made of a round-bottomed 96-well microplate sold under the name Costar® by the company Corning (reference: 7007).

Preferably, the 3D culture support is not a flat-bottomed microplate.

In a first embodiment, said fibroblasts derived from the dermal papilla and/or from the sheath are seeded on a 2D culture support not permitting cell adhesion, at a density of at least 14 000 cells/cm², preferably at a density of between 14 000 cells/cm² and 47 600 cells/cm² and better still between 23 500 cells/cm² and 24 500 cells/cm².

In a second preferred embodiment, said fibroblasts derived from the dermal papilla and/or from the sheath are seeded on a round-bottomed 3D microplate culture support not permitting cell adhesion, at a density of at least 3000 cells/cm², preferably of at least 9000 cells/cm² and better still at a density of between 9000 cells/cm² and 20 000 cells/cm².

The process for the in vitro preparation of a dermal papilla equivalent according to the invention may also comprise a preliminary step of amplifying said fibroblasts derived from the dermal papilla and/or fibroblasts derived from the connective tissue sheath in a nutritive culture medium A; said nutritive culture medium A comprising at least serum, in particular fetal calf serum.

Preferably, the culture support used for this amplification step is a support treated to permit cell adhesion. These supports are those which are conventionally used for cell culture and are therefore well known to a person skilled in the art.

Preferably, said nutritive culture medium A used in said amplification step comprises from 500 to 2000 mg/l of amino acids, from 15 to 35 mg/l of vitamins, from 2500 to 4500 mg/l of glucose, from 7500 to 9500 mg/l of inorganic salts, from 5% to 30% by volume of fetal calf serum, and optionally from 50 to 200 μg/ml of antibiotics and/or of antimycotics.

Preferentially, said nutritive culture medium A used in said amplification step comprises from 750 to 1800 mg/l of amino acids, from 20 to 30 mg/l of vitamins, from 3000 to 4000 mg/l of glucose, from 8000 to 9000 mg/l of inorganic salts, from 10% to 25% by volume of fetal calf serum, and optionally from 100 to 180 μg/ml of antibiotics and/or of antimycotics.

The amino acids present in said culture medium A used in said amplification step are preferably chosen from glycine, L-alanyl-glutamine, L-arginine hydrochloride, L-cystine dihydrochloride, L-histidine hydrochloride hydrate, L-isoleucine, L-leucine, L-lysine hydrochloride, L-methionine, L-phenylalanine, L-serine, L-threonine, L-tryptophan, L-tyrosine, L-valine, L-alanine, L-asparagine, L-aspartic acid, L-glutamic acid and L-proline, and mixtures thereof.

The vitamins present in said culture medium A used in said amplification step are preferably chosen from choline chloride, calcium D-pantothenate, folic acid, niacinamide, pyridoxine hydrochloride, riboflavin, thiamine hydrochloride, i-inositol, and mixtures thereof.

The organic salts present in said culture medium A used in said amplification step are preferably chosen from calcium chloride (CaCl₂), magnesium sulfate (MgSO₄), iron nitrate (Fe(NO₃).9H₂O), potassium chloride (KCl), sodium bicarbonate (NaHCO₃), sodium chloride (NaCl), sodium dihydrogen phosphate (NaH₂PO₄.4H₂O), and mixtures thereof.

Examples of antibiotics present in said medium A that may be mentioned include penicillin, streptomycin, and mixtures thereof.

An example of an antimycotic present in said medium A that may notably be mentioned is amphotericin B.

In a particularly preferred embodiment, said nutritive culture medium A used in said amplification step comprises from 70% to 80% by volume of DMEM Glutamax medium, from 5% to 25% of fetal calf serum, from 50 to 90 mg/l of nonessential amino acids, and optionally from 50 to 200 μg/ml of antibiotics and/or of antimycotics. Better still, said nutritive culture medium A comprises 78% by volume of DMEM Glutamax medium, 20% of fetal calf serum, 60 to 80 mg/l of nonessential amino acids, and optionally from 100 to 180 μg/ml of antibiotics and/or of antimycotics.

The nonessential amino acids present in said culture medium A used in said amplification step are chosen from L-alanine, L-asparagine, L-aspartic acid, L-glutamic acid, L-proline and L-serine, and mixtures thereof.

In a particularly preferred embodiment, the process for preparing an in vitro dermal papilla equivalent according to the invention also comprises, prior to the step of culturing said fibroblasts, the following preliminary steps:
a. isolating an anagen-phase hair follicle from a scalp sample;
b. recovering the fibroblasts of the dermal papilla and/or of the connective tissue sheath by means of microdissection of the dermal papilla and/or of the connective tissue sheath;
c. performing an amplification of said dermal papilla fibroblasts and/or of said connective tissue sheath fibroblasts in a nutritive culture medium A consisting of DMEM Glutamax supplemented with 20% by volume of fetal calf serum (FCS), 50 to 90 mg/l of nonessential amino acids, and optionally 50 to 200 μg/ml of antibiotics and/or of antimycotics.

The present invention also relates to an in vitro dermal papilla equivalent which may be obtained by means of the process according to the invention as described above.

Preferably, the dermal papilla equivalent according to the invention is characterized in that it has a positive alkaline phosphatase activity, and optionally a positive expression of proteins chosen from BMP2 (Bone morphogenetic protein 2), SFRP2 (secreted frizzled related protein 2), CORIN, SOX2, VCAN (Versican) and/or PLC (Perlecan).

The dermal papilla equivalent according to the invention is preferably characterized in that it is constituted of cells resulting from a step of culturing fibroblasts derived from the dermal papilla and/or from the connective tissue sheath on a support comprising a serum-free nutritive culture medium B for a period of time that is sufficient to allow said fibroblasts to detach from said support and to group together to form at least one spheroid; said support not permitting cell adhesion; said culture support is chosen from 2D or 3D round-bottomed microplate culture supports.

The dermal papilla equivalent according to the invention is in particular a sphere with a diameter ranging from 100 μm to 250 μm. Preferably, the dermal papilla equivalent according to the invention is a sphere approximately 200 μm in diameter.

The enzymatic activity of the alkaline phosphatase may be measured, for example, using the NBT/BCIP alkaline phosphatase kit sold by the Roche laboratory (ref.: 11 681 451 001 on Oct. 30, 2017) in which the BCIP (5-bromo-4-chloro-3-indolyl phosphate, toluidine salt), the alkaline phosphatase substrate, will first be dephosphorylated and then oxidized to give a blue-colored product.

The protein expression of the markers BMP2, SFRP2, CORIN, SOX2, VCAN and PLC may be measured, for example, by means of the immunofluorescence technique, this technique moreover being well known to those skilled in the art.

### Process for Preparing a Hair Follicle Equivalent

The present invention also relates to the use of an in vitro dermal papilla equivalent according to the invention and of proliferative epithelial cells for preparing an in vitro hair follicle equivalent.

The present invention also relates to a process for the in vitro preparation of a hair follicle equivalent, comprising at least one step of culturing proliferative epithelial cells in the presence of at least one dermal papilla equivalent according to the invention for a period of time that is sufficient to allow differentiation of said proliferative epithelial cells into keratinocytes positive for the markers K85 and K35.

The proliferative epithelial cells are preferably chosen from the matrix cells and the germ cells as defined previously, and mixtures thereof.

In a particularly preferred embodiment, the proliferative epithelial cells used in the context of the present invention are the matrix cells.

### Step of Culturing the Proliferative Epithelial Cells in the Presence of at Least One in Vitro Dermal Papilla Equivalent

The proliferative epithelial cells seeded onto at least one dermal papilla equivalent according to the invention are cultured at 37° C. and at 5% CO₂, in a defined medium, such as the one described by Philpott (1993).

In particular, the proliferative epithelial cells are cultured in the same nutritive culture medium as that used for preparing the dermal papilla equivalents according to the invention, in particular the serum-free nutritive culture medium B.

In a particularly preferred embodiment, said dermal papilla equivalent is obtained by means of the process for preparing a dermal papilla equivalent as mentioned above in the section “Process for preparing a dermal papilla equivalent”.

The step of culturing said proliferative epithelial cells in the presence of at least one dermal papilla equivalent according to the invention is preferably performed on the 2D or 3D supports as defined previously, the surface of which does not permit cell adhesion.

The proliferative epithelial cells are preferably seeded at high density. Preferably, the proliferative epithelial cells are seeded at a density of at least 2000 cells/cm², preferably at least 6000 cells/cm², and better still at a density of between 6000 and 10 000 cells/cm².

Preferably, said proliferative epithelial cells are cultured for a period of time that is sufficient to allow the formation of at least one tubular structure, also known as a follicular organoid.

For the purposes of the present invention, the term “tubular structure” or “follicular organoid” means the budding observed after culturing of at least one dermal papilla equivalent according to the invention combined with proliferative epithelial cells chosen from the matrix cells and/or the germ cells.

Said proliferative epithelial cells are cultured in the presence of a dermal papilla equivalent for at least 3 days, preferably for at least 5 days, better still between 5 and 20 days, preferentially between 5 and 15 days.

In a particular embodiment, the process for preparing the hair follicle equivalent according to the invention comprises a step of adding, to the nutritive culture medium B, growth factors chosen from proteins of the WNT family, in particular the WNT3A protein, proteins of the BMP family, in particular the BMP2 and BMP4 proteins, and mixtures thereof. The addition of said growth factors is performed between the 1^{st} day and the 5^{th} day starting from the seeding of the proliferative epithelial cells.

The present invention also relates to an in vitro hair follicle equivalent which may be obtained according to the abovementioned process.

More particularly, the in vitro hair follicle equivalent is characterized in that it is constituted of a dermal papilla equivalent having a positive alkaline phosphatase activity and of keratinocytes that are positive for the markers K85 and K35 and optionally Ki67.

The alkaline phosphatase activity is measured according to the method mentioned above in the section “Process for preparing a dermal papilla equivalent”.

The labeling of the hair-specific keratins K85 and K35 and of the Ki67 protein, which is a cell proliferation marker, are performed according to the immunofluorescence method.

The hair follicle equivalent in particular has a solid tubular structure with a diameter of between 100 μm and 250 μm, and a length of at least 500 μm, preferably a length ranging from 500 μm to 2500 μm, better still from 1500 μm to 2500 μm.

### Preliminary Amplifying Step

The process for the in vitro preparation of a hair follicle equivalent according to the invention may comprise, prior to the step of culturing proliferative epithelial cells in the presence of at least one dermal papilla equivalent according to the invention, a preliminary step of amplifying proliferative epithelial cells in the presence of an effective amount of a ROCK inhibitor.

The proliferative epithelial cells such as the matrix cells and the germ cells are extracted by microdissection of the hair follicle and are amplified according to the technique of Rheinwald and Green (Cell, vol. 6, 331-344, 1975) by culturing on a feeder support constituted of fibroblasts in a suitable medium known to those skilled in the art, in the presence of growth factors, notably amino acids, serum, cholera toxin, insulin, triiodothyronine and pH buffer solution. In particular, such a culture medium may notably contain at least one mitogenic growth factor for keratinocytes (for example epidermal growth factor (EGF) and/or keratinocyte growth factor (KGF), in particular KGF), insulin, hydrocortisone and optionally an antibiotic (e.g.: gentamycin, amphotericin B) to which a ROCK inhibitor has been added.

The ROCK inhibitors may be chosen from: Y27632, Ripasudil, Fasudil, Thiazovivin, and mixtures thereof.

Advantageously, said medium may also comprise serum or a pituitary extract, for example of bovine origin, epinephrine, transferrin and/or nonessential amino acids.

The fibroblasts used for this culture will more preferentially be 3T3 fibroblasts. 3T3 fibroblasts are well known to those skilled in the art. It is a fibroblast cell line that has been known since 1962. “3T3” means “3-day transfer, inoculum of 3×10⁵ cells”.

The proliferative epithelial cell culture is preferably a culture on fibroblasts (preferentially 3T3 fibroblasts), the proliferation of which has been stopped beforehand, preferentially by having previously irradiated them (for example with gamma rays) or having previously treated them with mitomycin. Mitomycin (in particular mitomycin C) blocks the proliferation of these cells without, however, preventing them from producing nutritive substances useful for keratinocyte proliferation.

According to the invention, the effective amount of the ROCK inhibitor, in particular Y27632, is between 1 and 100 μM, preferably between 5 and 25 μM and preferably 10 μM.

According to the invention, the epithelial cells are cultured in the presence of the ROCK inhibitor, in particular Y27632, for at least 2 days and preferably for at least 3 days.

Preferably, the cells are placed in culture at a cell density of between 1000 and 4000 cells/cm² and preferably at a density of 3000 cell/cm².

### Uses

Given that the in vitro dermal papilla and hair follicle equivalents have, respectively, most of the features of a dermal papilla and of a hair follicle in vivo, they may be used as implants, optionally combined with skin substitutes.

The in vitro dermal papilla and hair follicle equivalents according to the invention will therefore also have applications for the preparation of implants and/or of skin substitutes for treating a skin disorder, such as a burn, a healing defect or canities.

A therapeutic effect is defined as a return to the normal state of pilosity, whether totally or partially.

For the purposes of the invention, prophylactic treatment is recommended if the subject has a prior condition for hair loss, such as a familial predisposition.

The conditions of a reduced amount of hair may be the result of alopecia, hereditary baldness, scars, burns or accidental injuries.

Thus, a subject of the present invention is also a hair follicle equivalent according to the invention for the prophylactic or therapeutic treatment of a state of reduced pilosity.

Another of its subjects is a hair follicle equivalent according to the invention for the treatment of alopecia.

Moreover, the present invention also relates to a dermal papilla equivalent for the prophylactic or therapeutic treatment of a state of reduced pilosity or alopecia.

### Process for Screening for New Active Agents

The dermal papilla and hair follicle equivalents according to the invention make it possible notably to establish growth kinetics for bodily hair or head hair and therefore to perform any study requiring numerous hairs that are alive and as complete as possible in an in vivo context such as the study of the hair cycle and of the factors capable of influencing this cycle, ranging up to the study of active agents which promote hair growth, of active agents which make it possible to combat hair loss or else of active agents which slow down the growth of bodily hair.

Thus, the present invention also relates to the use of an in vitro hair follicle equivalent according to the invention, for identifying compounds which modulate the growth of bodily hair and/or head hair.

Moreover, the present invention also relates to a process for screening for at least one compound which modulates the growth of bodily hair and/or head hair, comprising a step (a) of bringing said test compound into contact with an in vitro hair follicle equivalent according to the invention, then a step (b) of analyzing the effect of said compound on at least one parameter of the in vitro hair follicle equivalent and a step (c) of selecting the compound which modifies said parameter.

Preferably, to perform step (a), the modulating test compound is applied topically, for example formulated in conventional topical formulations or else introduced into the culture medium.

Step (b) may, in particular, be performed by analyzing the expression, the production and/or the activity of markers associated with the quality and/or with the homeostasis of the hair follicle, for instance epidermal and/or dermal markers, such as structural proteins. As an example of structural proteins, mention may be made of the hair keratins.

For this, the effect of the product on the growth of the hair shaft will be analyzed in step (b) of the screening process.

Step (b) of analyzing the effect of the product will preferentially be a comparison of at least one parameter measured on the hair follicle equivalent brought into contact with the test product with that or those measured on a control hair follicle equivalent cultured under the same conditions but which has not received the test product.

Step (c) of selecting the product which modifies the parameter of the hair follicle equivalent will be performed as a function of a criterion determined beforehand.

The modification of this parameter may be a stimulation, a decrease or a total or partial inhibition of the expression, of the production and/or of the activity of said markers and/or of the growth of the hair shaft.

The criterion for selecting said product will, for example, be that this product has a stimulatory or inhibitory effect on the parameter measured.

The hair follicle equivalent according to the invention may also be used in automated processes for screening cosmetic, pharmaceutical or dermatological compounds for identifying novel active agents.

In addition, the invention also relates to a process for screening for at least one compound which modulates the growth of bodily hair and/or head hair, comprising a step (a) of bringing said test compound into contact with an in vitro dermal papilla equivalent according to the invention, then a step (b) of analyzing the effect of said compound on at least one parameter of the in vitro dermal papilla equivalent and a step (c) of selecting the compound which modifies said parameter.

### FIGURES

FIG. 1: Localization of the matrix cells and of the fibroblasts used in the context of the present invention.

FIG. 2: Localization of the germ cells.

FIG. 3: Amplification of the fibroblasts derived from a dermal papilla in the nutritive culture medium A.

FIG. 4: Production of a dermal papilla equivalent after culture of the amplified fibroblasts in the serum-free nutritive culture medium B.

FIG. 5: T+1 day after seeding of the matrix cells on the dermal papilla equivalents.

FIG. 6: Formation of a tubular structure responsible for the formation of a hair follicle equivalent (T+4 days starting from the seeding of the matrix cells on the dermal papilla equivalents).

FIG. 7: Growth of the hair follicle equivalent (T+10 days starting from the seeding of the matrix cells on the dermal papilla equivalents).

FIG. 8: Strongly positive alkaline phosphatase activity of a dermal papilla equivalent according to the invention.

FIG. 9: Hair follicle showing positive labeling for the markers K35, K85 and Ki67.

FIG. 10: Comparative WO 2017/055358: T+10 days starting from the seeding of the matrix cells.

FIG. 11: Comparative outside the invention: dermal papilla obtained according to the process described in example 2 of WO 2009/118283 (magnification ×10, D6)

FIG. 12: Dermal papilla equivalent according to the invention obtained according to example 1 on a 2D culture support (magnification ×10, D6).

FIG. 13: Comparative outside the invention: hair follicle in cyst form obtained according to the process described in example 3 of WO 2009/118283 (D3)

FIG. 14: Comparative outside the invention: culture of fibroblasts obtained from dermal papilla on a 2D culture support for cell culturing (i.e. support permitting cell adhesion).

FIG. 15: Dermal papilla equivalent according to the invention obtained according to example 1 on a 3D round-bottomed microplate culture support (D2).

FIG. 16: Comparative outside the invention: hair follicle obtained in 3D culturing on collagen gel.

In the description and the examples that follow, unless otherwise indicated, the ranges of values written in the form “between . . . and . . . ” include the stated lower and upper limits.

For the purposes of the present invention, the term “at least one” should be understood, unless otherwise indicated, as meaning “one or more”.

The examples given below are presented as nonlimiting illustrations of the invention.

### EXAMPLE 1—PREPARATION OF A DERMAL PAPILLA EQUIVALENT ACCORDING TO THE INVENTION

### Experimental Protocol

i. Microdissection of the Dermal Papilla Fibroblasts

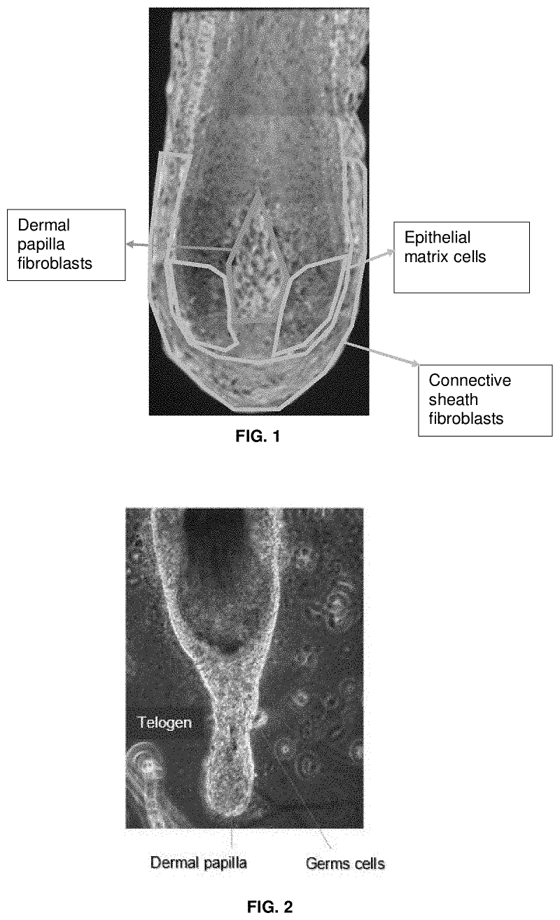

The fibroblasts derived from the dermal papilla were sampled according to the process which follows: anagen-phase hair follicles, dissected from a facelift, are placed in a Petri dish containing a minimum culture medium supplemented with 2% of antibiotic and nonessential amino acids. (FIG. 1).

ii. Culture Conditions:
nutritive culture medium A for fibroblast amplification:

The nutritive culture medium A for fibroblast amplification has the following composition:

| | Final concentrations | Volumes |
|---|---|---|
| DMEM Glutamax | 78% by volume | 390 ml |
| Gibco No. 31966047 | | |
| Fetal calf serum (FCS) | 20% by volume | 100 ml |
| FetalClone No. SH30072.03 | | |
| Nonessential amino acids | 1% by volume | 5 ml |
| Gibco No. 11140-035 | | |
| Antibiotics/Antimycotics | 1% by volume | 5 ml |
| Gibco No. 15240-062 | | |

Details of the commercial media used for the preparation of the culture medium A

DMEM Glutamax medium (Gibco No. 31966047)

| Compounds | Concentration (mg/l) |
|---|---|
| Glycine | 30.0 |
| L-Alanyl-L-Glutamine | 862.0 |
| L-Arginine hydrochloride | 84.0 |
| L-Cystine dihydrochloride | 63.0 |
| L-Histidine hydrochloride hydrate | 42.0 |
| L-Isoleucine | 105.0 |
| L-Leucine | 105.0 |
| L-Lysine hydrochloride | 146.0 |
| L- Methionine | 30.0 |
| L-Phenylalanine | 66.0 |
| L-Serine | 42.0 |
| L-Threonine | 95.0 |
| L-Tryptophan | 16.0 |
| L-Tyrosine | 72.0 |
| L-Valine | 94.0 |
| Choline chloride | 4.0 |
| Calcium D-pantothenate | 4.0 |
| Folic acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Calcium chloride (CaCl2•2H2O) | 264.0 |
| Iron nitrate (Fe(NO3)3•9H2O) | 0.1 |
| Magnesium sulfate (MgSO4•7H2O) | 200.0 |
| Potassium chloride (KCl) | 400.0 |
| Sodium bicarbonate (NaHCO3) | 3700.0 |
| Sodium chloride (NaCl) | 6400.0 |
| Sodium dihydrogen phosphate | 141.0 |
| (NaH2PO4•2H2O) | |
| D-Glucose (Dextrose) | 4500.0 |
| Phenol Red | 15.0 |

Nonessential amino acids Gibco No. 11140-035

| Compounds | Concentration (mg/l) |
|---|---|
| Glycine | 750.0 |
| L-Alanine | 890.0 |
| L-Asparagine | 1320.0 |
| L-Aspartic acid | 1330.0 |
| L-Glutamic acid | 1470.0 |
| L-Proline | 1150.0 |
| L-Serine | 1050.0 |

Antibiotics/Antimycotics Gibco No. 15240-062

| Compounds | Concentration (mg/l) | |
|---|---|---|
| Penicillin | 10 000 | U/ml |
| Streptomycin | 10 000 | μg/ml |
| Amphotericin b | 25 | μg/ml |

nutritive culture medium B for preparing the dermal papilla equivalents

The nutritive culture medium B for preparing the dermal papilla equivalents has the following composition:

| | |
|---|---|
| Final concentrations | Volumes |

| | | | | |
|---|---|---|---|---|
| Williams E (glutamine-free) | 98% | by volume | 489 | ml |
| Gibco No. A12176-01 | | | | |
| L-glutamine | 2 | mM | 5 | ml |
| Antibiotics/Antimycotics | 1% | by volume | 5 | ml |
| Gibco No. 15240-062 | | | | |
| Insulin | 10 | μg/ml | 500 | μl |
| Hydrocortisone | 10 | ng/ml | 10 | μl |

Details of the commercial media used for the preparation of the culture medium B

Williams E medium (glutamine-free) (Gibco No. A12176-01)

| Compounds | Concentration (mg/l) |
|---|---|
| Glycine | 50.0 |
| L-Alanine | 90.0 |
| L-Arginine | 50.0 |
| L-Asparagine hydrate | 20.0 |
| L-Aspartic acid | 30.0 |
| L-Cysteine | 40.0 |
| L-Cystine dihydrochloride | 26.07 |
| L-Glutamic acid | 50.0 |
| L-Histidine | 15.0 |
| L-Isoleucine | 50.0 |
| L-Leucine | 75.0 |
| L-Lysine hydrochloride | 87.46 |
| L-Methionine | 15.0 |
| L-Phenylalanine | 25.0 |
| L-Proline | 30.0 |
| L-Serine | 10.0 |
| L-Threonine | 40.0 |
| L-Tryptophan | 10.0 |
| Disodium salt of L-tyrosine | 50.65 |
| dihydrate | |
| L-Valine | 50.0 |
| Ascorbic Acid | 2.0 |
| Biotin | 0.5 |
| Choline chloride | 1.5 |
| Calcium D-pantothenate | 1.0 |
| Ergocalciferol | 0.1 |
| Folic acid | 1.0 |
| Menadione sodium bisulfate | 0.01 |
| Niacinamide | 1.0 |
| Pyridoxal hydrochloride | 1.0 |
| Riboflavin | 0.1 |
| Thiamine hydrochloride | 1.0 |
| Vitamin A (acetate) | 0.1 |
| Vitamin B12 | 0.2 |
| alpha•Tocopherol phos. | 0.01 |
| sodium salt | |
| i-Inositol | 2.0 |
| Calcium chloride (CaCl2) (anhydrous) | 200.0 |
| Copper sulfate (CuSO4•5H2O) | 1.0E−4 |
| Iron sulfate (FeSO4•7H2O) | 1.0E−4 |
| Magnesium sulfate (MgSO4) (anhydrous) | 97.67 |
| Manganese sulfate (MnSO4•H2O) | 1.0E−4 |
| Potassium chloride (KCl) | 400.0 |
| Sodium bicarbonate (NaHCO3) | 2200.0 |
| Sodium chloride (NaCl) | 6800.0 |
| Anhydrous sodium dihydrogen | 140.0 |
| phosphate (NaH2PO4) | |
| Zinc sulfate (ZnSO4•7H2O) | 2.0E−4 |
| D-Glucose (Dextrose) | 2000.0 |
| Glutathione (reduced) | 0.05 |
| Methyl linoleate | 0.03 |
| Sodium pyruvate | 25.0 |

Antibiotics/Antimycotics Gibco No. 15240-062 (cf. above)

### Culture-Amplification of the Dermal Papilla Fibroblasts

The dermal papilla located in the bulb region of the follicle is pinpointed under the microscope. The dermal papilla is microdissected using a scalpel and needles and is then placed in a culture dish containing the nutritive culture medium A as described above. (FIG. 3).

### Culture-Preparation of the Dermal Papilla Equivalent

After amplification of the fibroblasts in monolayer culture, the fibroblasts are trypsinized and then deposited in a Petri dish not treated for cell culture (Falcon® bacteriological Petri dish, Corning, ref.: 351007) at high density (for example 23 800 cells per cm²), in the serum-free nutritive culture medium B as described above.

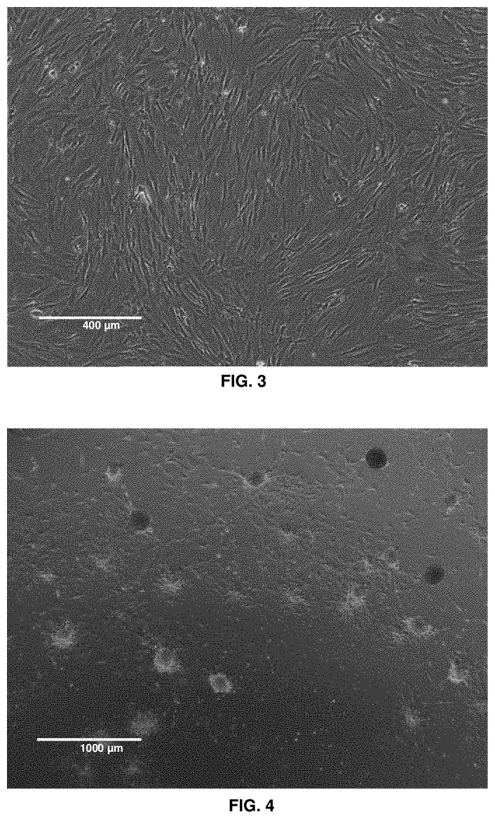

The fibroblasts migrate in the Petri dish and group together to form clusters, then cell aggregates, and finally detach from the support so as to form dermal papilla spheroids or equivalents after 5 days of culture. (FIGS. 4 and 12).

The dermal papillae obtained are in spherical form with a diameter of about 200 μm.

Labeling of the alkaline phosphatase enzymatic activity is performed using the NBT/BCIP alkaline phosphatase kit (Roche ref.: 11 681 451) in which BCIP (5-bromo-4-chloro-3-indolyl phosphate, toluidine salt), the alkaline phosphatase substrate, will first be dephosphorylated then oxidized to give a blue-colored product.

A vivid dark violet color shows a strongly positive alkaline phosphatase enzymatic activity. (FIG. 8).

Dermal papilla equivalents according to the invention obtained according to the same process as that of Example 1 were also prepared, replacing:
the 2D support (Falcon® bacteriological Petri dish, Corning) with the round-bottomed 96-well 3D microplate support sold under the name Costar® by the company Corning;
the fibroblast seeding density of 23 800 cells/cm² with 9375 cells/cm².

The dermal papillae obtained are also of spherical shape with a diameter of about 200 μm (see FIG. 15) and have a highly positive alkaline phosphatase enzymatic activity.

Conclusion: The dermal papillae according to the invention thus obtained do indeed have the morphological and functional features of an in vivo dermal papilla.

### EXAMPLE 2—PREPARATION OF A HAIR FOLLICLE EQUIVALENT ACCORDING TO THE INVENTION

### Experimental Protocol

i. Microdissection of the Matrix Cells

The hair follicles are extracted from a surgical residue of scalp. Said residue is first cut into 5 mm² portions and then sectioned using a scalpel between the dermis and the hypodermis.

The follicles are extracted using ophthalmic surgery forceps and are then sectioned just above the papilla with a scalpel. The bulb is then recovered. At this stage, the bulb comprises two compartments: the dermal compartment (dermal papilla and connective tissue sheath) and the matrix cells which form a cell mass. (FIG. 1).

The epithelial part is separated from the dermal part using perfusion needles.

ii. Culture Conditions

The culture conditions have three main components:
The base medium:

Unless otherwise indicated, all of the media and buffers used in the examples are described in Bell et al. 1979, (P.N.A.S. USA, 76, 1274-1278), Asselineau and Prunieras, 1984, (British J. of Derm., 111, 219-222) or Asselineau et al., 1987, (Models in dermato., vol. III, Ed. Lowe & Maibach, 1-7).

The DMEM medium+10% FCS+7F (called G7F medium) has the following composition:

| | Final concentrations |
|---|---|
| DMEM | 500 ml |
| Fetal calf serum (FCS) | 10% |
| L-Glutamine | 2 mM |
| Sodium pyruvate | 1 mM |
| Penicillin - Streptomycin | Penicillin 20 U/ml |
| | Streptomycin 20 μg/ml |
| Fungizone | Penicillin 10 U/ml |
| | Streptomycin 10 μg/ml |
| | Amphotericin-B 25 ng/ml |
| Epidermal growth factor (EGF) | 10 ng/ml |
| Cholera toxin | 10⁻¹⁰ M |
| Hydrocortisone | 0.4 μg/ml |
| Adenine hydrochloride | 1.8 × 10⁻⁴ M |
| Triiodothyronine (T3) | 2 × 10⁻⁹ M |
| Human transferrin | 5 μg/ml |
| Bovine insulin | 5 μg/ml |

The culture supplements: 10 μM of Y27632.
The adhesion surface: the matrix cells adhere and proliferate in the Green base medium in the presence of a feeder layer of murine 3T3 fibroblasts arrested in the cell cycle by mitomycin treatment.

### Culture-Amplification of the Matrix Cells

After microdissection, the matrix cell clumps are deposited in Petri dishes 60 mm in diameter, seeded beforehand with a feeder layer, preferably with 40 000 irradiated 3T3 cells/cm², and covered with the complete culture medium, for example the G7F medium+10 μM Y27632; a culture of matrix cells at 70% confluence is obtained.

### Culture-Preparation of the Hair Follicle Equivalent

In order to generate the in vitro hair follicle equivalents, the cells are recovered at the subconfluent stage by enzymatic treatment. The cells are then seeded in bacteriological Petri dishes not treated for cell culture (Falcon® bacteriological Petri dish, Corning, ref.: 351007), containing beforehand the dermal papilla equivalents, at a density of 6000 cells/cm², in the serum-free nutritive culture medium B as described in Example 1.

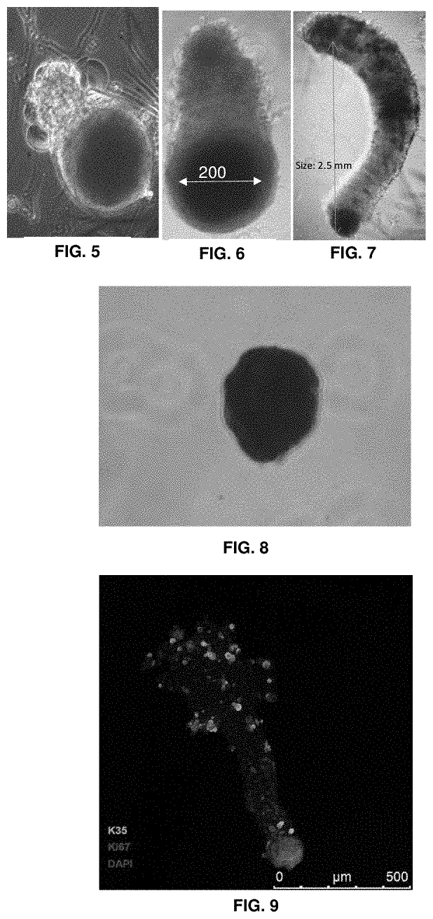

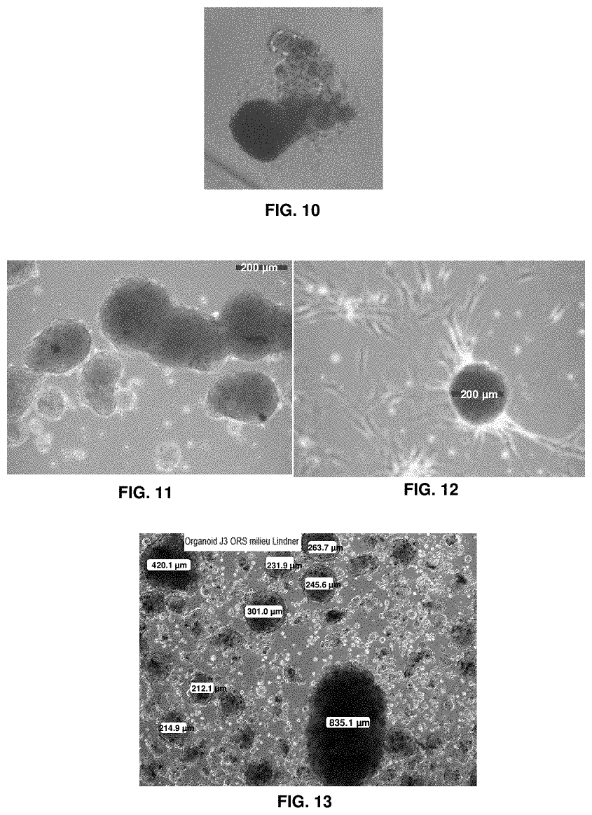

The matrix cells adhere only at the level of the dermal papilla spheroids and proliferate. (FIGS. 5 and 6).

After 6 days of coculture, hair follicle equivalents are seen to appear, in particular having the following morphological features: solid tubular structure about 2500 micrometers in diameter. (FIGS. 6 and 7).

The labeling of the hair-specific keratins K85 and K35, and also a cell proliferation marker, such as Ki67, is performed by fluorescence immunolabeling. (FIG. 9).

Conclusion: The hair follicles according to the invention thus obtained do indeed have the morphological and functional features of an in vivo hair follicle.

### EXAMPLE 3

### Comparative Outside the Invention: Dermal Papilla Obtained According to the Process Described in Example 2 of WO 2009/118283

1) Materials and Methods:

The preparation of the dermal papilla equivalent was performed according to the preparation protocol described in example 2 of WO 2009/118283.

Preparation of the DMEM (+) medium:
500 ml of DMEM Glutamax (Invitrogen No. 31966)
5 ml of nonessential amino acids (NEAA) (Gibco No. 11140-035)
50 μl of ascorbic acid at 100 mM (Sigma No. A8960), i.e. 2.9 mg/l final
5 ml of Insulin-Transferrin-Sodium Selenite (ITS) (Fisher Scientific No. 10524233)
400 mg/l BSA

Culture support: Flat-bottomed 6-well ULA (ultra low attachment) 3D plate.

Cell density: 6660 cells/cm².

Cells: fibroblasts obtained from a dermal papilla (passage P6).

2) Results: (See FIG. 11)

Conclusion: very heterogeneous cell aggregates of different sizes with irregular edges are observed.

### EXAMPLE 4

### Comparative Outside the Invention: Dermal Papilla Obtained According to the Process Described in Example 3 of WO 2009/118283

1) Materials and Methods:

The preparation of the hair follicle equivalent was performed according to the preparation protocol described in example 3 of WO 2009/118283.

Culture Medium:
DMEM (+) for the papilla equivalents (DMEM+2% BSA+ITS+Vit C)
Then KSFM for the DP/MHN/ORS mixed culture

Culture support: Flat-bottomed 6-well ULA (ultra low attachment) plate.

Cell Density:
For the preparation of the dermal papilla: fibroblasts obtained from a dermal papilla: 500 000 DP/F75 i.e. 6660 DP/cm²;
For the preparation of the hair follicle: 250 000 ORS/6 wells, i.e. 26 300 ORS/cm²+25 000 MHN/6 wells, i.e. 2630 MHN/cm².

Cells:
Fibroblasts obtained from a dermal papilla (passage P6).
Melanocytes M597 (passage P4)
Keratinocytes from ORS IB (passage P3)

2) Results (See FIG. 13)

Conclusion: cell aggregates (cysts) are observed without evolution toward a hair follicle.

### EXAMPLE 5

### Comparative Outside the Invention: Culture of Fibroblasts Obtained from Dermal Papilla on a 2D Culture Support for Cell Culturing (i.e. Support Permitting Cell Adhesion)

1) Materials and Methods:

The preparation of the dermal papilla equivalent was performed according to the preparation protocol described in example 1, replacing the Falcon® bacteriological Petri dish culture support, Corning, ref.: 351007 with a Petri dish culture support for cell culturing (i.e. permitting cell adhesion).

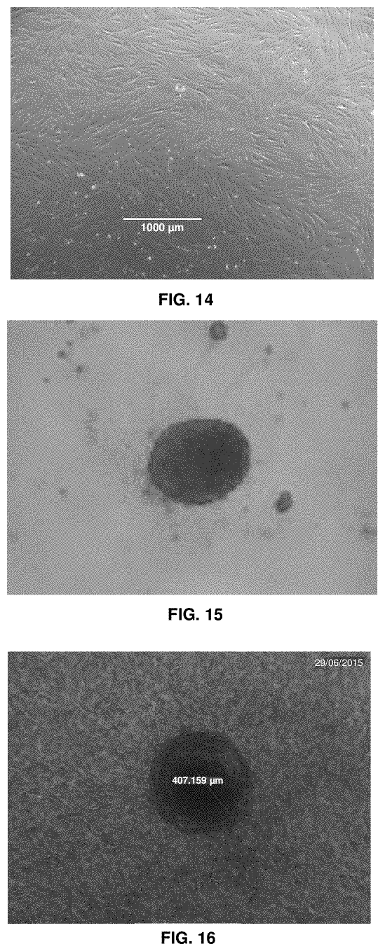

2) Results: (See FIG. 14)

Conclusion: No formation of dermal papilla is observed using a culture support permitting cell adhesion.

### EXAMPLE 6

### Comparative Outside the Invention: Dermal Papilla Obtained in a Culture Medium Containing Serum as Described in Higgins et al. (“Modelling the Hair Follicle Dermal Papilla Using Spheroid Cell Cultures”)

1) Materials and Methods:

Comparative Outside the Invention:
3D Culture support: U-bottomed 96-well ULA plate
Culture medium: DMEM medium+10% FCS
Cell density: 9375 cells/cm²

Dermal Papilla Equivalent According to the Invention:
3D Culture support: U-bottomed 96-well ULA plate
Culture medium: Williams medium (serum-free)
Cell density: 9375 cells/cm²

In order to measure the RNA expression of the alkaline phosphatase, Versican, and SFRP2 secreted frizzled related protein 2, which are markers of the enzymatic activity of the dermal papilla, the probes ALPL-Hs01029144_m1, VCAN-Hs00171642_m1, and SFRP2-Hs00293258_m1 were used.

Expression variations

| | DP P5 3D | DP P5 3D |
|---|---|---|
| | DMEM + 10% | WILLIAMS E |
| | FCS (outside the | (according to the |
| Probes | invention) | invention) |
| ALPL-Hs01029144_m1 | 1.00 | 1.75 |
| VCAN-Hs00171642_m1 | 1.00 | 3.61 |
| SFRP2-Hs00293258_m1 | 1.00 | 2.18 |

Conclusion:
For the dermal papilla equivalent according to the invention, overexpression is observed for the markers ALPL (alkaline phosphatase), VCAN (versican) and SFRP2 (secreted frizzled related protein 2), which are known as markers of inductivity of the enzymatic activity of the dermal papilla.
For the comparative outside the invention, underexpression is observed for the markers ALPL (alkaline phosphatase), VCAN (versican) and SFRP2 (secreted frizzled related protein 2), which are known as markers of inductivity of the enzymatic activity of the dermal papilla.

### EXAMPLE 7

### Comparative Outside the Invention: Hair Follicle Obtained in 3D Culturing on Collagen Gel

1) Materials and Methods:

A first step of manufacturing of a spheroid comprising fibroblasts obtained from dermal papilla and from proliferative epithelial cells (matrix cells) was performed in DMEM medium+10% serum, in a U-bottomed 96-well ULA plate.

A second step was then performed, which consists in integrating the spheroids into a collagen gel (lattice), observation being performed on D14.

2) Results: (See FIG. 16)

Conclusion: No formation of hair follicles is observed in the collagen gel; a cyst is observed without apical structure formation.

1. A process for the in vitro preparation of a dermal papilla equivalent, comprising at least one step of culturing fibroblasts derived from the dermal papilla and/or from the connective tissue sheath on a support comprising a serum-free nutritive culture medium B for a period of time that is sufficient to allow said fibroblasts to detach from said support and to group together to form at least one spheroid; the surface of said support used not permitting cell adhesion; said culture support is chosen from 2D or 3D round-bottomed microplate culture supports.

2. The process as claimed in claim 1, in which said fibroblasts are seeded on said 2D culture support at a density of at least 14 000 cells/cm².

3. The process as claimed in claim 1, in which said fibroblasts are seeded on said 3D culture support at a density of at least 3000 cells/cm².

4. The process as claimed in claim 1, in which said nutritive culture medium B comprises from 500 to 1500 mg/l of amino acids, from 2 to 18 mg/l of vitamins, from 1500 to 4500 mg/l of glucose, from 8750 to 10 000 mg/l of inorganic salts, from 2 to 20 μg/ml of insulin, from 2 to 60 ng/ml of hydrocortisone, and optionally from 50 to 200 μg/ml of antibiotics and/or of antimycotics.

5. The process as claimed in claim 1, in which said fibroblasts are cultured for at least 3 days.

6. The process as claimed in claim 1, in which the surface of said support is neutral and hydrophobic.

7. The process as claimed in claim 1, also comprising, prior to the step of culturing said fibroblasts, the following preliminary steps:
a. isolating an anagen-phase hair follicle from a scalp sample;
b. recovering the fibroblasts of the dermal papilla and/or of the connective tissue sheath by means of microdissection of the dermal papilla and/or of the connective tissue sheath;
c. performing an amplification of said dermal papilla fibroblasts and/or of said connective tissue sheath fibroblasts in a nutritive culture medium A consisting of DMEM Glutamax supplemented with 20% by volume of fetal calf serum (FCS), 50 to 90 mg/l of nonessential amino acids, and optionally 50 to 200 μg/ml of antibiotics and/or of antimycotics.

8. An in vitro dermal papilla equivalent which may be obtained by means of the process as claimed in claim 1.

9. The in vitro dermal papilla equivalent as claimed in claim 8, which has positive alkaline phosphatase activity.

10. A method preparing an in vitro hair follicle equivalent which comprises using the in vitro dermal papilla equivalent as claimed in claim 8 and of proliferative epithelial cells.

11. A process for the in vitro preparation of a hair follicle equivalent, comprising at least one step of culturing proliferative epithelial cells in the presence of at least one dermal papilla equivalent as defined in claim 8 for a period of that is time sufficient to allow differentiation of said proliferative epithelial cells into keratinocytes positive for the markers K85 and K35.

12. The process as claimed in claim 11, in which the proliferative epithelial cells are seeded at a density of at least 2000 cells/cm².

13. The process as claimed in claim 11, in which the proliferative epithelial cells are cultured for at least 3 days.

14. The process as claimed in claim 11, also comprising a preliminary step of amplifying said proliferative epithelial cells in the presence of an effective amount of a ROCK inhibitor.

15. An in vitro hair follicle equivalent which may be obtained by means of the process as claimed in claim 11.

16. The in vitro hair follicle equivalent as claimed in claim 15, which is constituted of a dermal papilla equivalent having a positive alkaline phosphatase activity and of keratinocytes positive for the markers K85 and K35.

17. The in vitro hair follicle equivalent as claimed in claim 15, wherein it has a solid tubular structure with a diameter ranging from 100 to 250 μm, and a length ranging from 500 to 2500 μm.

18. The in vitro hair follicle equivalent as claimed in claim 15, for the prophylactic or therapeutic treatment of a state of reduced pilosity.

19. The in vitro hair follicle equivalent as claimed in claim 15, for the treatment of alopecia.

20. A process for identifying compounds which modulate the growth of bodily hair and/or head hair which comprises using the in vitro hair follicle equivalent as claimed in claim 15.

21. A process for screening for at least one compound which modulates the growth of bodily hair and/or head hair, comprising a step (a) of bringing said test compound into contact with an in vitro hair follicle equivalent as claimed in claim 15, then a step (b) of analyzing the effect of said compound on at least one parameter of the in vitro hair follicle equivalent and a step (c) of selecting said compound which modifies said parameter.
